(19) United States
(12) Patent Application Publication
(10) Pub. No.: US 2025/0032705 A1
(43) Pub. Date: Jan. 30, 2025

(54) Medical Safety Needle Assembly

(71) Applicant: Design 33 Inc., Cheyenne, WY (US)

(72) Inventor: John Simon Forber, Liguge (FR)

(73) Assignee: Design 33 Inc., Cheyenne, WY (US)

(21) Appl. No.: 18/716,112

(22) PCT Filed: Dec. 5, 2022
(86) PCT No.: PCT/IB2022/000678

§ 371 Date: Jun. 3, 2024

(30) Foreign Application Priority Data
Dec. 3, 2021 (FR) FR2112968

(51) Int. Cl. A61M 5/158

(52) U.S. Cl. CPC A61M 5/158; A61M 2005/1587

(57) ABSTRACT

Medical safety needle assembly comprising a medical needle (14) having a distal and a proximal end and a stem extending there between, the needle having a tip at its distal end, the proximal end of the needle being connected to a tubing (15) with a connection hub for connecting the tubing (15) to a syringe (30), the connection being able to resist tensile loads, and a base plate (10) the needle (14) extending slidably through the base plate (10) and having an inserted position with the needle tip extending in distal direction from the base plate (10) and a retracted position with the needle tip being in a position inside a cavity of the base plate, characterized in that the needle comprises a first attachment element at its proximal side of the base plate (10), and the connection hub comprises a second attachment element (31) being able to engage with the first attachment element in order to pull the needle (14) through the base (10) plate into its retracted position.

The present invention relates to a medical safety needle assembly.

Implantable vascular access systems are used extensively in the medical field to facilitate the performance of repeated injections of therapeutic treatments inside the body of a patient. The vascular access system is generally comprised of a vascular access port that is attached to a catheter. The vascular access port generally consists of a housing with an elastomeric septum exposed on the upper surface. Beneath the elastomeric septum is a reservoir that can be accessed by the tip of a needle when it passes the elastomeric septum. The vascular access port has a tubular elongated member extending from the reservoir that allows attachment of the catheter.

The vascular access system is implanted into the body of a patient. The distal tip of the catheter is disposed at a predetermined location where the therapeutic agent is delivered, generally the entrance of the right atrium just beyond the superior vena cava. Once the vascular access system is implanted, the care giver can insert the tip of the needle through the skin, the elastomeric septum and into the reservoir space to deliver the treatment. In order to minimize the height of the needle extending from the skin, the needles are bent approximately 90° so that they extend minimally above the skin of the patient.

The tip of the needle has a bend close to the principle bevel of the needle. This bend is such that once formed the principle bevel is approximately parallel to the axis of the unbent portion of the cannula. The generally accepted terminology for this type of needle tip is a “Huber” tip. When a needle of this conception is forced through the elastomeric septum, it creates just a slit in the septum. When the needle is removed, the slit surfaces return in contact and under the radial compression of the septum and the intrinsic characteristics of the elastomeric material a seal is maintained. If a regular hypodermic needle was inserted into the elastomeric septum there is an elevated risk that the needle may core particles from the septum. This could potentially impair the septum and/or liberate the particles that could obstruct the catheter or be released into the vasculature of the patient.

As with any needle type device, there exists the risk of inadvertent needle sticks, that generally occur when the needle is removed from the patient prior to disposal of the needle. This risk is particularly significant in the case of Huber needles being removed from a vascular access system. This is because the force to remove a Huber needle from a port is high due the fact that the needle is, being compressed radially by the elastomeric septum of the vascular access port. Also the care giver generally needs to stabilize the vascular access port with the fingers of the opposite hand at a location close to the exit site of the needle.

To address the problem of inadvertent needle sticks, various safety devices have been designed. One safety device is described in U.S. Pat. No. 5,755,694 that-discloses a needle base disposed over a segment of the needle at its proximal end, comprising two generally flat wings made of a flexible plastic material, with one hinge connecting each of the wings to the needle base. Upon removal of the needle from the patient, the wings flex against the moveable members that keeps them adjacent to the needle until the needle is completely removed from the skin of the patient after which the wings surround the needle to prevent the tip from making contact outside the needle base.

Many advancements have been made in needle devices for use with vascular access ports to reduce the risk of needle sticks. Additionally there is another potential problem. When the needle is inserted through the septum, it occupies a volume corresponding to the volume of the needle and liquid therein that extends below the elastomeric septum. When the needle is removed from the vascular access port, this volume of the needle and liquid within is removed without replacement, causing an equivalent volume of venous blood to draw into the distal end of the catheter. This infiltration of blood is suspected to cause or potentially contribute to thrombus formation at the tip of the catheter, which could block the flow of fluid through the catheter. The risk is dependent on the inner diameter of the catheter as the equivalent volume of blood drawn back equates to a short column of blood in a large catheter, whereas in a small diameter catheter, the equivalent volume could be quite long and hence increase the potential for thrombus formation. If this were to occur it could result in early removal of the vascular access port and catheter. This early removal is costly, invasive, time consuming and otherwise completely undesirable.

One solution to this problem has been to implement a positive flushing technique. This technique consists of the medical practitioner inserting a syringe to the hub of the “Huber” needle and injecting heparinized saline into the port while simultaneously withdrawing the needle. The effect being, that the injected volume of liquid compensates for the volume of the needle that is being removed from the reservoir and the port. However this technique is quite cumbersome and complicated and in many cases cannot be done by a single care giver as many safety needles require two hands to operate. One such device that allows for positive flushing by one care giver is described in U.S. Pat. No. 7,927,317 that discloses a needle with a two part foldable needle cover. When the needle is in the patient, the two part cover is folded down. To remove the needle the care giver lifts the cover to the vertical position, then places the thumb on the top of the fixed part of the cover. The care giver then places the forefinger and middle finger on the flanges extending on the moveable part of the cover. In this position, the care giver pulls up on the moveable part of the cover that in turn pulls the needle up and into the fixed part of the cover. At the end of the travel, the moveable portion of the cover locks to the fixed part of the cover preventing the needle from moving downwards. Because only one hand is required for this operation it frees up the second hand to slowly inject liquid through the syringe as the first hand removes the needle.

This technique is effective, however this device is cumbersome and has several significant drawbacks. Firstly the size of the cover and mechanism is very large. The larger the device is above the skin, the higher the risk that the needle might be inadvertently hit and become dislodged from the patient. Also the fact that the device extends significantly above the skin of the patient is cosmetically undesirable. Additionally, during the removal of the device, the limited size of the device's contact pad and the force required to remove the needle can be very unconformable.

Accordingly, the object of the present invention is to provide a medical needle assembly that is easy to remove and that provides the possibility of performing a positive flushing during needle removal by one single person.

The object of the invention is achieved with a medical safety needle assembly comprising a medical needle having-a distal and a proximal end and a stem extending there between. The needle has a tip at its distal end, and the proximal end of the needle is connected to a tubing with a connection hub for connecting the tubing to a syringe, wherein the connection is able to resist tensile loads. The assembly further comprises a base plate. The needle extends slidably through the base plate and has an inserted position with the needle tip extending in distal direction from the base plate and a retracted position with the needle tip being in a position inside a cavity of the base plate.

The medical needle assembly of the invention is characterized in that the needle comprises a first attachment element at its proximal side of the base plate, and in that the connection hub comprises a second attachment element being able to engage with the first attachment element in order to pull the needle through the base plate into its retracted position.

The first attachment element of the needle may in particular be an eye, and the second attachment element of the connection hub a hook that may engage with the eye of the needle, so that the needle may be pulled back from its inserted position into its retracted position. However, the first and second attachment elements may be any kind of element that may engage with each other in a way that a force applied on the connection hub is transferred to the needle to bring it from the inserted into the retracted position. In particular, any form-locked or any force-locked connection may be used.

When the needle is in its inserted position, the distal side of the base plate is in contact with a patient's skin, while the needle tip may for example be inserted into a vascular access system. A liquid may be injected into the vascular access system when a syringe or another suitable device like an infusion pump is connected to the connection hub of the assembly.

A tubing, in particular a flexible tube, is attached between the proximal end of the needle and the connector hub that creates a continuous fluid path from the tip of the needle to the connector hub.

The connection hub may be a rigid part with a central lumen that extends through the length of the part.

The connection possibility provided by the connection hub shall be able to resist tensile loads. For this purpose, at one end of the lumen of the connector, the internal and external form and dimensions may for example conform to the ISO standard for Luer connectors (ISO 80369-7). In such case, a standard syringe with a corresponding Luer connector may be connected with the connection hub, and the connections resists tensile loads in the direction of the lumen of the connector.

The opposite end is dimensioned to allow for the bonding of the tubing.

The connector hub may preferably have one or more radial protrusions from the body of the part, the protrusions aide in applying the torque necessary to attach the connector hub to another connector, preferably a Luer connector.

The second attachment element of the connector hub, e.g. a hook, shall be able to engage with the first attachment element of the needle, e.g. an eye, so that both parts may be connected by a caregiver. For removal of the needle, a syringe may be connected to the connection hub, and the second attachment element may be attached to the first attachment element together with the syringe. The caregiver may then hold with one hand the base plate down on the skin of the patient and may pull the syringe upwards with the other hand, thus removing the needle from the patient. At the same-time, the caregiver may apply a light pressure on the syringe so that some liquid is inserted through the needle into the vascular access port or other device. The removal of the needle in this way does not create a backflow of blood into an implanted device but actively flushes it so that no blood is pulled into the thin vascular access system tube.

Preferably, a handle may be attached to the needle stem at its proximal side of the base plate. Such a handle may help the caregiver to introduce the needle into a patient's body, and such handle may in particular comprise the first attachment element, such as an opening forming an eye fora hook.

In a preferred embodiment of the present invention, the needle stem may have a 80°-90° bend at its proximal part above the base plate. The bend may be positioned such that it is close to the base plate when the needle is in its inserted position so that the assembly has a low profile and does not extend significantly above the skin of the patient.

In order to ensure straight insertion and removal of the needle, the handle may advantageously be attached close to the bend of the needle.

The handle may be movable between a position parallel to the base plate and a position vertical to the base plate, so that pressure in the distal direction of the needle may be applied for inserting the needle, and that tension close to the needle shaft may be applied for removing the needle by pulling on a syringe.

In an embodiment of the invention, the handle may consist of two parts that may move independently, e.g. in the form of two wings. The two parts of the handle may extend into opposite directions in their position parallel to the base plate. Both parts may also form a single handle when they are in the vertical position. Preferably, they may engage with each other in the vertical position, e.g. by means of suitable connection elements like e.g. interconnecting and releasable clips.

The assembly comprises preferably safety means that prevent the needle tip from leaving the cavity inside of the base plate. Such means are well known in the state of the art and not subject of this invention.

Various embodiments of the invention are described in greater detail in the following, using the attached figures:

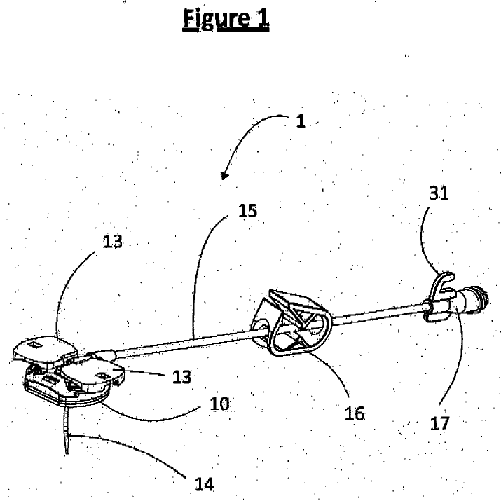

FIG. 1 is an isometric view of one embodiment of the present invention in the inserted needle position;

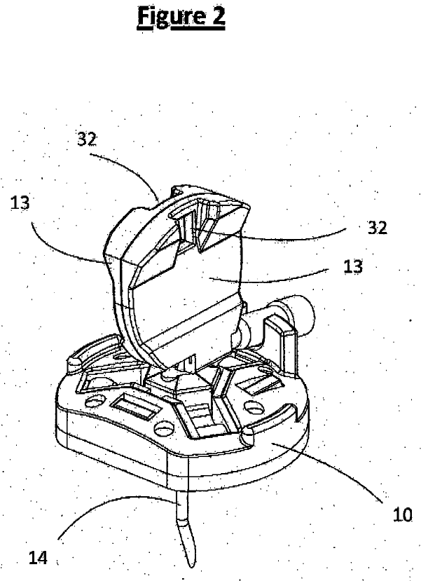

FIG. 2 is an isometric view of the base plate and the needle of the embodiment of FIG. 1 after insertion of the needle with the tubing and the connecting hub removed;

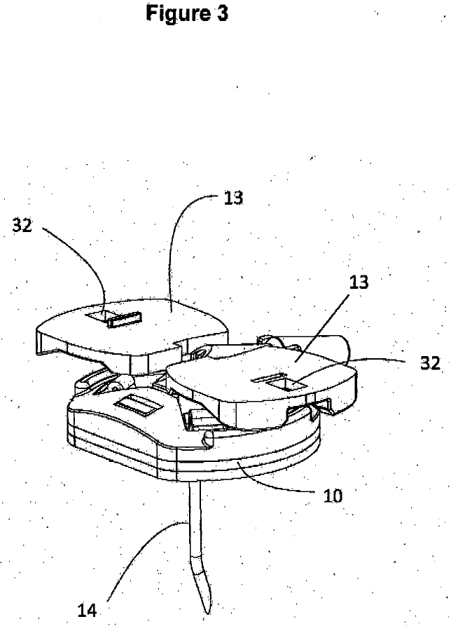

FIG. 3 shows the embodiment of FIG. 2 in the inserted needle position with the handle folded down to achieve a low profile;

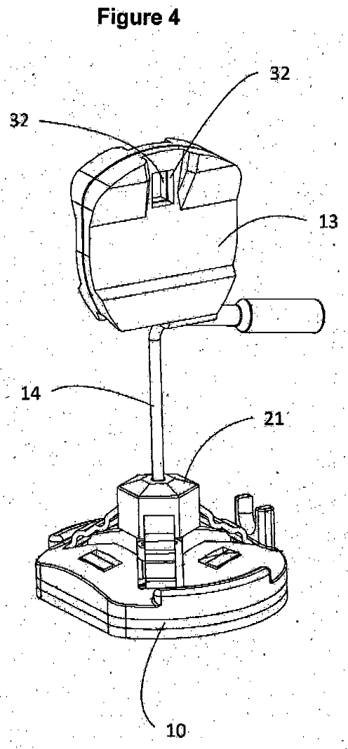

FIG. 4 is an isometric view of the base plate and needle of FIG. 3 in the retracted needle position;

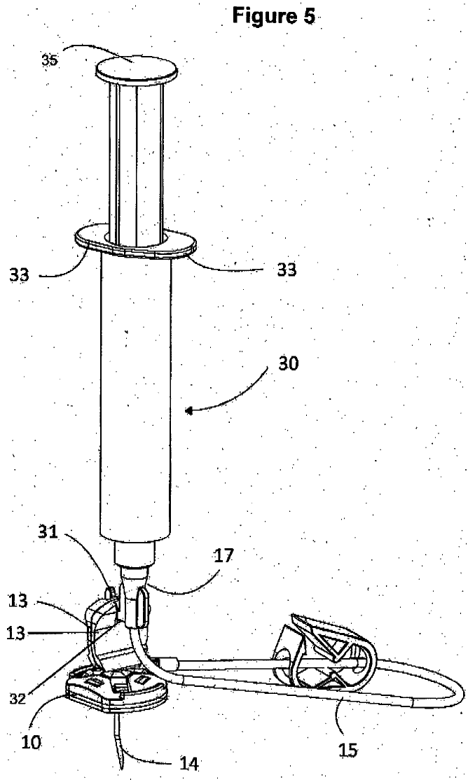

FIG. 5 is an isometric view the embodiment of the invention of the previous figures in the inserted needle position, prepared for needle removal with positive flushing;

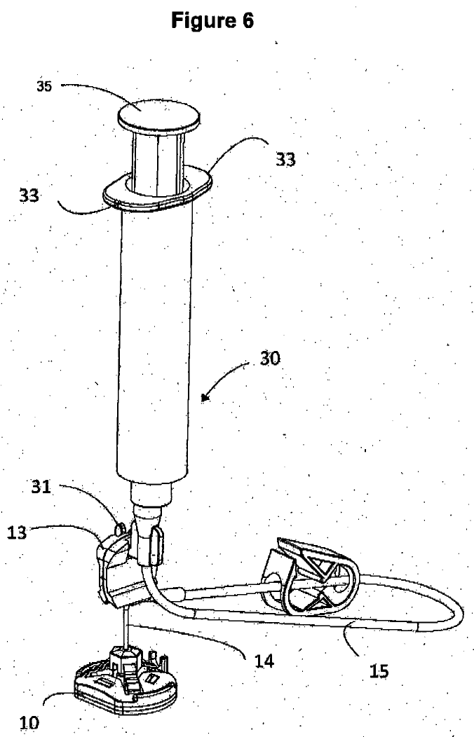

FIG. 6 is an isometric view of the assembly of FIG. 5 in the retracted needle position.

It will be further understood that the terms “comprises” and/or “comprising” when used in this specification, specify the presence of stated features, steps, operations, elements and/or components, but do not preclude the presence or addition of one or more other features, steps, operations, elements, components, and/or groups thereof.

FIG. 1 is an isometric view of one embodiment of the medical safety needle assembly 1 in accordance with the present invention in the inserted needle position. The assembly comprises a base plate 10, a handle 13 comprising two parts, a needle 14, a tubing 15 and a connecting hub 17, In addition, a clamp 16 may be positioned on the tubing 15. The handle 13 comprises two parts that are movable between a position parallel to the base plate 10 shown in the figure and a position vertical to the base plate. The two parts of the handle 13 have each an opening 32 that form an eye in the vertical position of the handle parts, so that the hook 31 of the connection hub 17 may engage therein.

FIG. 2 is a closer isometric view of the base plate 10 with the needle 14 in the inserted needle position. To insert the needle 14 in the patient, the two wing-shaped parts of the handle 13 are folded up in a position essentially vertical to the base plate 10 allowing the care giver the stability to push the needle down through the skin and into the reservoir of a vascular access system.

After insertion of the needle, the wings may be folded down to achieve a low profile, as is shown in FIG. 3.

FIG. 4 is an isometric view of the elements shown in FIG. 3, but in the retracted position of the needle. To achieve this position, the care giver uses two fingers of one hand to push on the cover of the base plate 10 to maintain the base plate in contact with the patient's skin. The two wing-shaped parts of the handle 13 are folded up in a position essentially vertical to the base plate 10, and the care giver pulls up on the handle 13 vertically, using two fingers of the opposite hand until the needle tip is enclosed in a cavity within the base plate. A safety mechanism prevents the needle from leaving the cavity within the base plate in either direction, further upwards or further downwards. This mechanism is not part of the present invention, and suitable solutions are well-known in the state of the art.

FIG. 5 is a an isometric view of a medical safety needle assembly in accordance with the previous figures in the inserted needle position to allow for positive flushing during needle removal. In the shown configuration the connector hub 17 is first attached to a syringe 30 containing fluid, generally heparinized physiological saline. The care giver folds two movable parts of the handle 13 into their position vertical to the base plate 10 so that the two openings 32 form an eye.

The caregiver then inserts the hook 31 on the connector hub 17 through the eye of the handle 13. The care giver uses two fingers of one hand to maintain the base plate assembly 10 in contact with the patient's skin: With the opposite hand, the care giver can hold the syringe 30 in the standard manner, the thumb on the piston and the index and middle fingers beneath the syringes flanges 33. The care giver can then pull up on the syringe 30 with the two fingers beneath the flanges 33 which will in turn pull up on the handle 13 and needle 14. At the same time slight pressure can be applied to the piston 35 during removal of the needle 14, achieving positive flushing during removal. It is obvious that depending on the care giver they can use this positive flushing technique or decide not use positive flushing and just pull the needle up on the handle 13 directly with the opposite hand,

FIG. 6 is an isometric corresponding to FIG. 4 but in the removed needle position, wherein the needle tip is enclosed in a cavity within the base plate 10 after retraction of the needle 14.

1. Medical safety needle assembly (1) comprising
a medical needle (14) having a distal and a proximal end and a stem extending there between,
the needle having a tip at its distal end,
the proximal end of the needle being connected to a tubing (15) with a connection hub (17) for connecting the tubing (15) to a syringe, the connection being able to resist tensile loads,
and a base plate (10)
the needle (14) extending slidably through the base plate (10) and having an inserted position with the needle tip extending in distal direction from the base plate (10) and a retracted position with the needle tip being in a position inside a cavity of the base plate,
characterized in that
the needle comprises a first attachment element (32) at its proximal side of the base plate (10),
and the connection hub (17) comprises a second attachment element (31) being able to engage with the first attachment element (32) in order to pull the needle (14) through the base (10) plate into its retracted position.

2. Safety needle assembly according to claim 1, characterized in that the first attachment element is an eye (32).

3. Safety needle assembly according to claim 1 or 2, characterized in that the second attachment element is a hook (31).

4. Safety needle assembly according to any one of claims 1 to 3, characterized in that a handle (13) is attached to the needle stem at its proximal side of the base plate (10).

5. Safety needle assembly according to claim 4, characterized in that the handle (13) comprises the first attachment element, in particular an opening (32) as an eye.

6. Safety needle assembly according to any one of claims 1 to 5, characterized in that the needle stem has a 80°-90° bend at its proximal part of the base plate.

7. Safety needle assembly according to any one of claims 4 to 6, characterized in that the handle (13) is attached close to the bend.

8. Safety needle assembly according to any one of claims 4 to 7, characterized in that the handle (13) or parts thereof are movable between a position parallel to the base plate (10) and a position vertical to the base plate.

9. Safety needle assembly according to any one of claims 4 to 8, characterized in that the handle (13) consists of two parts that may move independently.

10. Safety needle assembly according to claim 9, characterized in that the two parts of the handle (13) extend into opposite directions in their position parallel to the base plate (10).

11. Safety needle assembly according to claim 9 or 10, characterized in that both parts form a single handle when they are in the vertical position.

12. Safety needle assembly according to any one of claims 9 to 11, characterized in that both parts of the handle engage with each other in the vertical position.
